(19) United States
(12) Patent Application Publication
(10) Pub. No.: US 2011/0106125 A1
(43) Pub. Date: May 5, 2011

(54) SUGICAL CUTTING ATTACHMENT

(75) Inventors: Bradley Erik Steele, Memphis, TN (US); Jeetendra Subhash Bharadwaj, Memphis, TN (US)

(73) Assignee: WARSAW ORTHOPEDIC, INC., Warsaw, IN (US)

(21) Appl. No.: 12/608,414

(22) Filed: Oct. 29, 2009

(51) Int. Cl. A61B 17/3201

(52) U.S. Cl. 606/174

(57) ABSTRACT

A surgical cutting attachment that may include an elongated first member with a proximal end and a distal end including a first blade and an elongated second member with a proximal end and a distal end including a second blade. The second member may be positioned in a side-by-side orientation with the first member and may be connected to the first member at a pivot. A first set of apertures may extend through the first member and a second set of apertures may extend through the second member. A connector may be positioned on the proximal end of at least one of the first and second members. The first and second members may be movable between a first orientation with the first set of apertures aligned with the second set of apertures and the first blade spaced away from the second blade, and a second orientation with the first and second sets of apertures spaced away from each other and the first blade in proximity to the second blade. The first and second blades may form a scissor mechanism and the first and second set of apertures may form a shear mechanism. Methods of using the surgical cutting attachment are also disclosed.

### BACKGROUND

The present application is directed to devices and methods for cutting an elongated member and, more specifically, for a cutting attachment that includes both scissor and shear mechanisms.

Elongated members are often implanted into patients to repair hard tissue damage or correct defects in skeletal features. Examples include implanting rods to repair and reposition broken bones in arms and legs or repair broken features associated with joints. Other examples include the use of vertebral rods to treat degenerative defects in the spine or treat spinal curvature.

For various reasons, these elongated members are constructed from materials such as metals, including titanium, cobalt chrome, and stainless steel. These materials provide various structural aspects that facilitate treatment of the patient, but are difficult to work with. One example is the difficulty in cutting the elongated members to the various dimensions needed to suit the particular patient.

It is often necessary to cut the elongated member to the desired dimensions during the surgical procedure. Often, the cutting of the elongated member is done iteratively. The surgeon may make an initial measurement of the patient and then make an initial cut of the elongated member while it is outside of the patient's body. The surgeon may subsequently make additional measurements and may make additional cuts. Once the dimensions are approximately correct, the surgeon may then place the elongated member into the patient. Additional cuts may also be necessary, often to make minor modifications. These cuts may be performed while the elongated member is within the patient. Access to the elongated member while it is inside the patient, and adequate space to perform the cut may be limited.

### SUMMARY

The present application is directed to surgical cutting attachments and methods of using the attachment. The attachment may include an elongated first member with a proximal end and a distal end including a first blade and an elongated second member with a proximal end and a distal end including a second blade. The second member may be positioned in a side-by-side orientation with the first member and may be connected to the first member at a pivot. A first set of apertures may extend through the first member and a second set of apertures may extend through the second member. A connector may be positioned on the proximal end of at least one of the first and second members. The first and second members may be movable between a first orientation with the first set of apertures aligned with the second set of apertures and the first blade spaced away from the second blade, and a second orientation with the first and second set of apertures spaced away from each other and the first blade in proximity to the second blade. The first and second blades may form a scissor mechanism and the first and second set of apertures may form a shear mechanism.

The various aspects of the various embodiments may be used alone or in any combination, as is desired.

### BRIEF DESCRIPTION OF THE DRAWINGS

FIG. 1 is a perspective view of a cutting attachment constructed according to one embodiment.

FIG. 2 is a side view of a first member according to one embodiment.

FIG. 3 is a sectional view cut along line III-III of FIG. 2.

FIG. 4 is a perspective view of a second member according to one embodiment.

FIG. 5 is a side view of a second member according to one embodiment.

FIG. 6 is a sectional view cut along line VI-VI of FIG. 5.

FIG. 7 is a sectional view cut along line VII-VII of FIG. 5.

FIG. 8 is a side view of a cutting attachment according to one embodiment.

FIG. 9 is a top view of a cutting attachment according to one embodiment.

FIG. 10 is an end view of a proximal end of the cutting attachment according to one embodiment.

FIG. 11 is an exploded schematic diagram of a cutting attachment and a drive assembly according to one embodiment.

### DETAILED DESCRIPTION

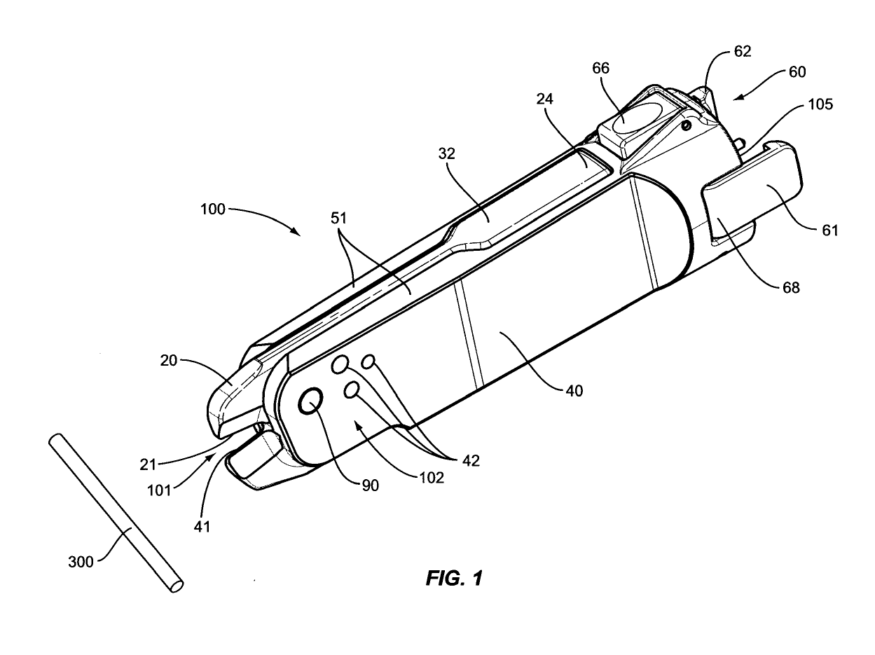

The present application is directed to a cutting attachment for cutting elongated members and methods of using the attachment. The cutting attachment features two different types of cutting mechanisms including a scissor mechanism and a shear mechanism. FIG. 1 includes a cutting attachment 100 with the scissor mechanism 101 positioned at a distal end, and the shear mechanism 102. Each mechanism 101, 102 can cut an elongated member 300. The cutting attachment 100 is formed by first and second members 20, 40 that are movable relative to each other. The scissor mechanism 101 is formed by a blade 21 on the first member 20 and corresponding blade 41 on the second member 40. The shear mechanism 102 is formed by one or more apertures in the first member 20 that correspond with one or more apertures 42 in the second member 40. The cutting attachment 100 further includes a connector 60 that attaches to a drive assembly for moving the members 20, 40 relative to each other during the various cutting processes.

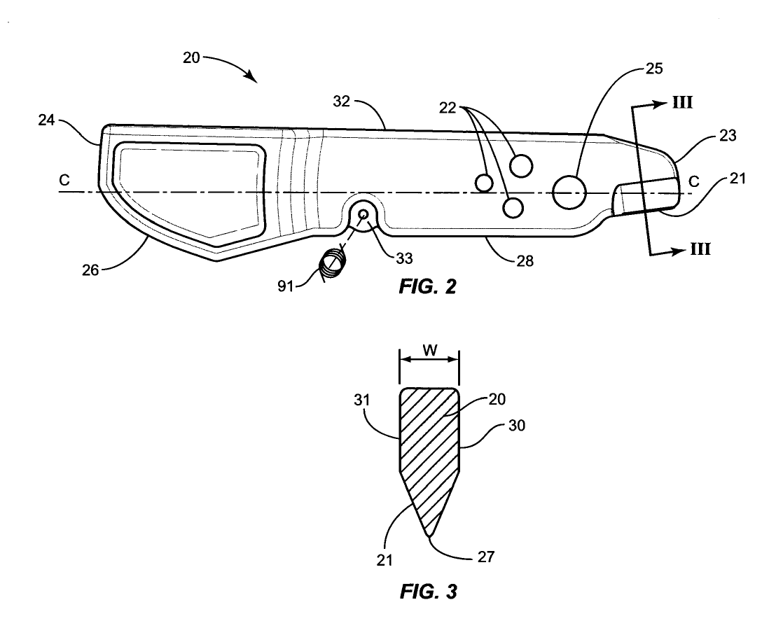

The first member 20 forms a first portion of the scissor and shear mechanisms 101, 102. FIG. 2 illustrates a first member 20 with an elongated shape with a length measured between a distal end 23 and a proximal end 24. The first member 20 also has a height measured between a top side 32 and a bottom side 28. A longitudinal centerline C extends along the first member 20 and through the ends 23, 24. The first blade 21 is positioned at the distal end 23. The first blade 21 is straight and is oriented at an acute angle relative to the centerline C. As illustrated in FIG. 3, the first blade 21 includes a tapered shape that terminates at a tip 27. The tip 27 is centered along a width W measured between opposing sides 30, 31 of the first member 20. As illustrated in FIG. 1, the width W may vary along the length between the distal and proximal ends 23, 24. FIG. 1 specifically includes the width W being less towards the distal end 23 and greater towards the proximal end 24.

One or more apertures 22 extend through the width W of the first member 20 to form a portion of the shear mechanism 102. The apertures 22 include sharp edges for cutting the member 300 during the shearing operation. FIG. 2 includes three apertures 22, although other embodiments may include a single aperture 22, two apertures 22, or more than three apertures 22. In embodiments with multiple apertures 22, each aperture 22 may be the same or may include different sizes to specifically accommodate different sizes of elongated members 300. The apertures 22 may also include different shapes and sizes. FIG. 2 specifically includes three circular apertures 22 with the first aperture 22 having a diameter of about 4.85 mm, the second aperture 22 having a diameter of about 5.6 mm, and the third aperture having a diameter of about 6.45 mm. These sizes are sized to fit tightly over members 300 of common diameters, such as 4.75 mm, 5.5 mm, and 6.35 mm. The tight fit of the member 300 within one of the apertures 22 leads to a cleaner shear cut by better holding the member 300 perpendicular to the cutting surfaces.

An additional aperture 25 extends through the first member 20 between the opposing sides 30, 31. The aperture 25 is sized to receive a member 90 to pivotally connect the first and second members 20, 40. As illustrated in FIG. 2, the aperture 25 may be positioned between the first blade 21 and the apertures 22.

The proximal end 24 is positioned towards and interacts with a drive assembly 200 as will be explained in greater detail below. The bottom edge 28 at the proximal end 24 includes an inclined ramp 26 that forms an acute angle with the centerline C of the first member 20. The inclined ramp 26 may be straight with the angle being constant along the length. Alternatively, the ramp 26 may include a curved shape, include sections with different slopes, and combinations thereof. The inclined edge 26 illustrated in FIG. 2 varies along the length. The ramp 26 is inclined away from the centerline C with the height of the first member 20 increasing away from the proximal end 24.

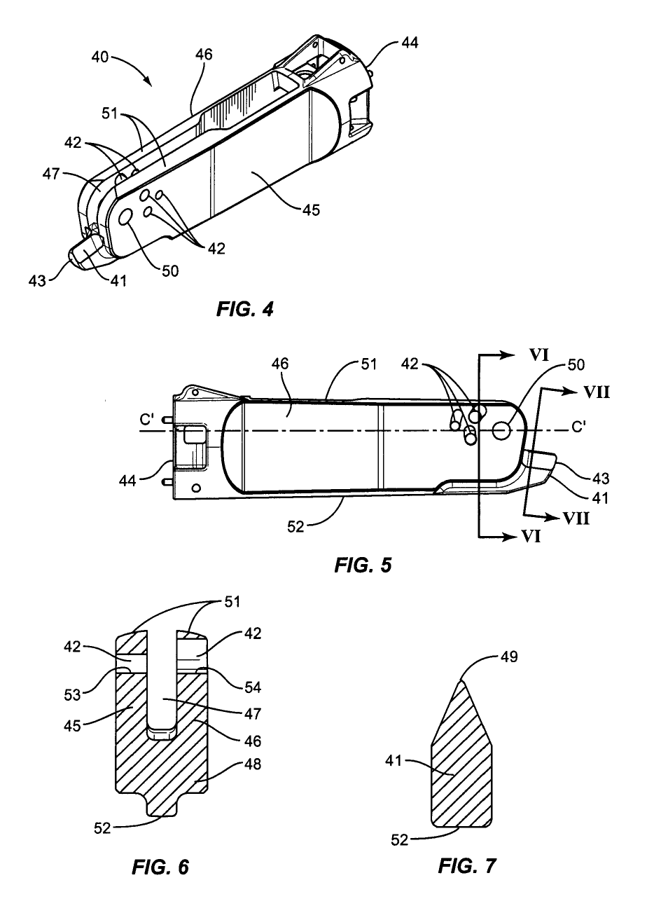

The second member 40 is movably connected to the first member 20. FIGS. 4 and 5 illustrate a second member 40 having an elongated shape with a length extending between a distal end 43 at the blade 41 and a proximal end 44. The length of the second member 40 may be greater than the first member 40. The second member 40 also includes a height measured between opposing top and bottom sides 51, 52.

The second member 40 may include opposing side members 45, 46 that are spaced apart a distance to form a channel 47 that receives the first member 20. The width of the channel 47 is sized to accommodate the width of the first member 20. As illustrated in FIGS. 1 and 4, the width of the channel 47 may be smaller at the distal end 43 and larger towards the proximal end 44 to accommodate the varying width of the first member 20. A base 48 may form a lower side of the channel 47 and connect the side members 45, 46 as illustrated in FIG. 6. The base 48 and side members 45, 46 enclose the channel 47 on three sides.

The blade 41 is positioned at the distal end 43 and extends outward beyond the side members 45, 46. The blade 41 is straight and is oriented at an acute angle relative to a centerline C′ of the second member 40. In one embodiment the angle θ is about 7.5°. This orientation causes the blade 41 to be aligned with the corresponding first blade 21 during the pivoting scissor motion. As illustrated in FIG. 7, the blade 41 includes a tapered shape that terminates at a tip 49. In one embodiment, the blade 41 is positioned along the width of the second member 40 with the tip 49 aligned with the channel 47 and between the side members 45, 46. In another embodiment, the tip 49 is aligned with one of the side members 45, 46.

One or more apertures 42 extend through all or a portion of the second member 40 to form a portion of the shear mechanism 102. The apertures 42 include sharp edges for cutting the member 300 during the shearing operation. FIG. 4 illustrates apertures 42 extending through both of the side members 45, 46. Each aperture 42 in the first side member 45 aligns with a corresponding aperture 42 in the second side member 46. This alignment allows for the elongated member 300 to be inserted during the shearing process as will be explained in detail below. The apertures 42 in the side member 45 may have the same or different shapes and or sizes as the opposing apertures 42 on the side member 46. In one embodiment, as illustrated in FIG. 5, the apertures 42 in the first member 45 are each substantially circular and the corresponding apertures 42 in the second member 46 are larger with each having a curved, swept circle shape (the shape may also be referred to as a “kidney bean” shape). In one embodiment, the apertures 42 in the second side member 46 are defined by a circle slightly larger than the corresponding apertures 42 in the first side member 45. These slightly larger apertures 42 are then swept radially through an angle, such as an angle of about 15°. As illustrated in FIGS. 5 and 6, the smaller circular apertures 42 in the first member 45 align with the ends of the larger apertures 42 in the second member 46. A side 53 of the aperture 42 in the first side member 45 aligns with a side 54 of the aperture 42 in the second side member 46. In one embodiment, the apertures 42 in the second side member 46 are sized slightly larger than the corresponding apertures 22 in the first member 20 to account for manufacturing variances in the size and location of the apertures 22, 42. The larger apertures 42 in the second side member 46 may not include sharp edges because they do not cut the member 300.

The number of apertures 42 in the members 45, 46 may vary. In one embodiment, three apertures 42 extend through each of the first member and second members 45, 46. In one embodiment, the apertures 42 in the side member 45 are round with one aperture having a diameter of about 5.0 mm, a second aperture having a diameter of about 5.75 mm, and a third aperture having a diameter of about 6.6 mm. The different sizes of apertures 42 facilitate elongated members of different cross-sectional sizes. In one embodiment, each aperture 42 in the side member 45 is substantially the same size and shape as the corresponding aperture 42 in the side member 46. In another embodiment, apertures 42 extend through just one of the side members 45 or 46.

In one embodiment, apertures 42 in the first side member 45 are circular and are slightly larger than the corresponding apertures 22 in the first member 20. The circular shapes of these apertures 42, 22 cause a more uniform pressure on the cutting edge formed on the edges of the apertures during the shearing action. Corresponding apertures 42 in the second side member 46 are larger those in the first side member 45. The apertures 42 in the first side member 45 include sharp edges while the apertures 42 in the second side member 46 do not. The member 300 is cut by the action between the aperture 42 in the first side member 45 and aperture 22. The larger apertures 42 in the second side member 46 allow the member 300 to translate during the shearing cut. The elongated member 300 passes through apertures 42 in second side member 46 with minimal, if any, contact. The member 300 is only cut at the intersection of apertures 42 in the first side member 45 and apertures 22 in the first member 20. If the apertures 42 in the second member 46 were not larger, the member 300 would be cut in two locations by the shear mechanism 102. During the shearing process, the apertures 22 move out of alignment with the apertures 42 in the first side member 45, and remain aligned with the apertures 42 in the second side member 46.

An additional aperture 50 extends through at least one of the side members 45, 46 to receive the member 90 to pivotally connect the first and second members 20, 40. In one embodiment, the aperture 50 extends through both side members 45, 46. In one embodiment, the aperture 50 is positioned between the blade 41 and the one or more apertures 42. The axis of the aperture 50 may be parallel with the axes of apertures 22 and 42.

The first and second members 20, 40 are pivotally connected together by the member 90 to form the scissor mechanism 101 and the shear mechanism 102. The first and second members 20, 40 are movable between an open orientation and a closed orientation. The open orientation as illustrated in FIGS. 1 and 8 positions the first member 20 relative to the second member 40 with the one or more apertures 22, 42 aligned to receive the elongated member 300. In the open orientation, the bottom side 28 of the first member 20 may rest against the base 48. The apertures 22, 42 form the shear mechanism 102 as will be explained in detail below.

The open orientation also positions the blade 21 of the first member 20 away from the blade 41 of the second member 40. As illustrated in FIG. 8, the tip 27 of the first member 20 is positioned at an angle β relative to a centerline X, and tip 49 of the second member 40 is positioned at an angle θ relative to the centerline X. The orientation of the tips 27, 49 provides for a space there between to receive the elongated member 300. The position of the blades 21, 41 outward at the distal end of the device 100 facilitates accessing and cutting the elongated member 300. This position is particularly beneficial when the elongated member 300 is positioned within the patient. The blades 21, 41 of the device 100 form the scissor mechanism 101.

In one embodiment of the open orientation, a majority of the first member 20 is nested within the channel 47 of the second member 40. As illustrated in FIGS. 1 and 8, the top side 32 of the first member 20 is aligned with or recessed below a top side 51 of the second member 40. Further, the heights of the first and second members 20, 40 measured between opposing top and bottom sides are substantially the same. This orientation provides a streamline shape that facilitates insertion into the patient.

In the closed orientation, the first member 20 is pivoted relative to the second member 40. This movement moves the one or more apertures 22 of the first member 20 away from the one or more apertures 42 of the second member 40. The apertures 22 in the first member 20 now overlap with the body of the second member 40, and the apertures 42 of the second member now overlap with body of the first member 20. The edges of the apertures 22, 42 contact against the elongated member 300 that extends through the paired apertures 22, 42 and thereby cuts the elongated member 300.

Movement to the closed orientation also moves the blade 21 of the first member 20 towards the blade 41 of the second member 40. The closed orientation may position the tips 27, 49 of the blades 21, 41 in contact, or in near contact. In one embodiment, the tips 27, 49 are positioned about 0.3 mm apart in the closed orientation. This movement to the closed orientation causes a scissor-action that cuts an elongated member 300 positioned between the blades 21, 41. The movement causes the proximal end 24 of the first member 20 to move out of the overlapping-alignment with the second member 40.

A biasing member 91 may extend between the first and second members 20, 40. The biasing member 91 may bias the first and second members 20, 40 to the first, open orientation. The biasing member 91 may also maintain the first member 20 within the channel 47 of the second member 40. In one embodiment, the biasing member 91 is connected to a notch 33 positioned along the bottom side 28 of the first member with an opposing section of the biasing member 91 connected to a latch component 92 inserted in the base of the second member 40.

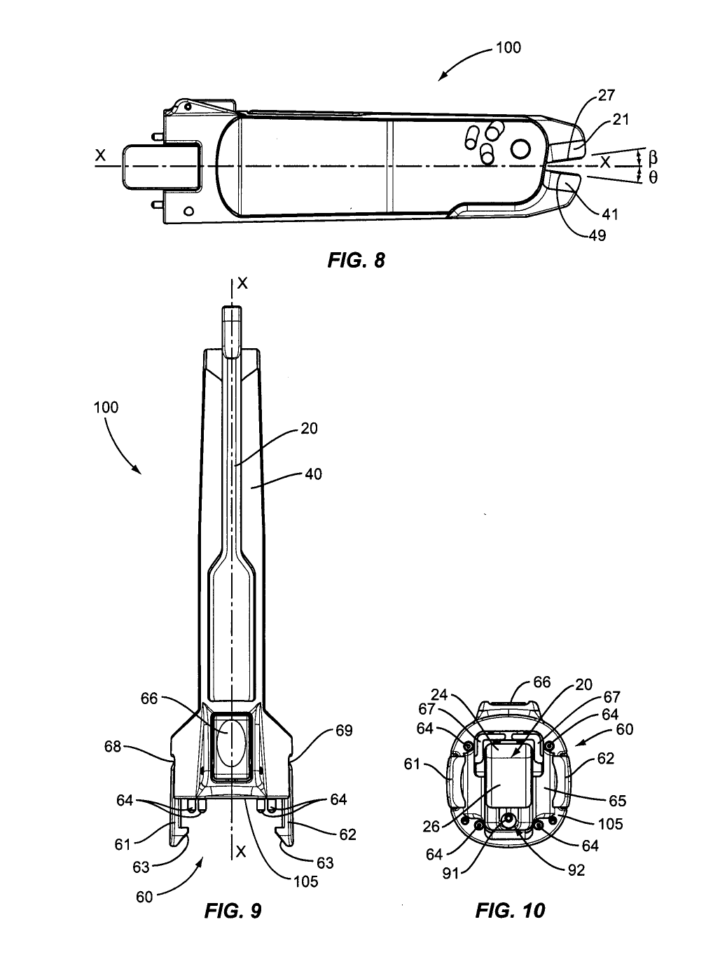

A connector 60 is positioned at the proximal end 105 of the cutting attachment 100 to attach to a drive assembly 200. The connector 60 may be isolated to just one of the first and second members 20, 40, or may be positioned on both members 20, 40. FIGS. 9 and 10 illustrate a connector 60 that includes a pair of opposing wings 61, 62. Each of the wings 61, 62 is pivotally connected to the second member 40 at ends 68, 69 respectively. The wings 61, 62 are movable between a locked position as illustrated in FIGS. 9 and 10 and an unlocked position with the wings pivoted outward from ends 68, 69. The locked position includes the wings 61, 62 substantially parallel to the centerline X of the attachment 100. The unlocked position includes the ends of the wings 61, 62 opposite from the ends 68, 69 being far away form the centerline X. Ramped tabs 63 may be positioned at the ends of the wings 61, 62 to connect with the drive assembly 200.

One or more projections 64 may extend outward from the proximal end 105. The projections 64 may be substantially straight and include a tapered end. The number and position of the projections 64 may vary. FIGS. 9 and 10 include a total of four projections 64. A cavity 65 is formed in the proximal end 105 to receive the drive assembly 200. The proximal end 24 of the first member 20 is exposed in the cavity 65 for contacting against the drive assembly 200. As specifically illustrated in FIG. 10, the inclined ramp 26 is positioned and exposed in the cavity 65.

An engagement button 66 is positioned on the exterior of the attachment 100. The button 66 is operatively connected to connectors 67 to control the movement of the wings 61, 62. Depression of the button 66 moves the connectors 67 and thereby moves the wings 61, 62 to the unlocked position. A biasing member (not illustrated) may engage and bias the button 66 against depression. The biasing member causes the button 66 and the connected wings 61, 62 to return to the locked position when the force is removed from the button 66. The biasing member also applies a force to maintain the wings 61, 62 locked to the drive assembly 200.

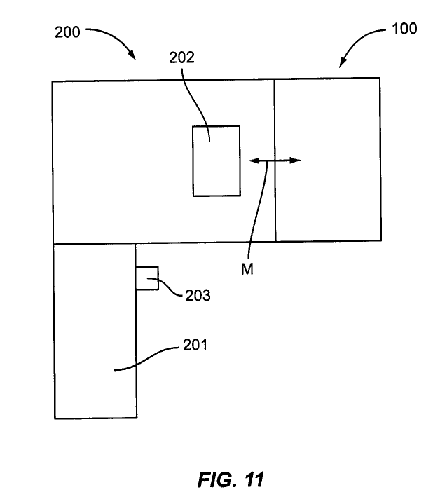

FIG. 11 illustrates a schematic view of the cutting attachment 100 connected to a drive assembly 200. The cutting attachment 100 is connected by the connector 60 as explained above. The drive assembly 200 may include a handle 201 that extends outward from a main body to facilitate handling by the user.

The drive assembly 200 may include a drive member 202 that is movable as illustrated by arrow M between an unengaged position and an engaged position. The engaged position includes the drive member 202 operatively connected with the cutting attachment 100 to move the first and second members 20, 40 between the first and second orientations. In one embodiment, the drive member 202 moves outward from the drive assembly 200 and engages with the inclined ramp 26 of the first member 20. The engagement causes the first member 20 to pivot about the member 90 and move relative to the second member 40 from the first orientation to the second orientation. The drive member 202 may then move towards an unengaged position away from the cutting attachment 100 and moving the first member 20 back to the first orientation.

The cutting attachment 100 may be used with a variety of different drive assemblies 200. Examples of drive assemblies include those disclosed in U.S. patent application Ser. No. 12/104,648 entitled Surgical Prosthetic Device Cutting Tool filed Apr. 17, 2008, and U.S. patent application Ser. No. 12/559,182, entitled Surgical Tool filed on Sep. 14, 2009. Both of these applications are hereby incorporated by reference in their entireties.

In use, the cutting attachment 100 is initially connected to a drive assembly 200. This may include initially depressing the button 66 and moving the wings 61, 62 to the unlocked position to receive the drive assembly 200. Once the two members are positioned together, the button 66 may be released and the wings 61, 62 move to the locked position to connect with the drive assembly 200. In another embodiment, the drive assembly 200 can be moved against the cutting attachment 100. The drive assembly 200 forces the wings 61, 62 outward such that the drive assembly 200 seats against the cutting attachment 100. The wings 61, 62 are biased inward and automatically lock against the drive assembly 200. Once assembled, the user can activate a switch 203 that moves the cutting attachment 100 between the first and second orientations.

Initially, the combined device is positioned in the first orientation. The user can then either use the scissor mechanism 101 to cut the elongated member 300 between the blade 21, 41, or the shear mechanism 102 to cut the elongated member 300 within one of the sets of corresponding apertures 22, 42. In many instances, the scissor mechanism 101 is used for cutting the elongated member 300 when it is positioned within the patient, and the shear mechanism 102 is used for cutting the elongated member 300 when it is away from the patient.

In the embodiments described above, the second member 40 is formed by first and second members 45, 46. In another embodiment, the second member 40 is formed by a single side member (similar to the first member 20). The single second member 40 is pivotally connected with the first member 20 and able to move between the open and closed orientations in a similar manner to the embodiment described above. The scissor mechanism 101 is formed between a blade on the single second member 40 and the blade 21 of the first member 20. The shearing mechanism 102 is formed between one or more apertures in the single second member 40 and the corresponding one or more apertures 42 in the first member 20.

The cutting attachment 100 may be used on living patients for the treatment of various disorders, such as the insertion of a vertebral rod to treat spinal disorders. The cutting attachment 100 may also be used in a non-living situation, such as with a cadaver, model, and the like. The non-living situation may be for one or more of testing, training, and demonstration purposes.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. A surgical cutting attachment to cut an elongated member comprising:
an elongated first member with a proximal end and a distal end including a first blade;
an elongated second member with a proximal end and a distal end including a second blade, the second member positioned in a side-by-side orientation with the first member and connected to the first member at a pivot;
a first set of apertures extending through the first member and each including a different size;
a second set of apertures extending through the second member;
a connector positioned on the proximal end of at least one of the first and second members;
the first and second members movable between a first orientation and a second orientation, the first orientation including the first set of apertures aligned with the second set of apertures and the first blade spaced away from the second blade, and a second orientation with the first and second set of apertures spaced away from each other and the first blade in proximity to the second blade;
the first and second blade forming a scissor mechanism and the first and second set of apertures forming a shear mechanism.

2. The surgical cutting attachment of claim 1, wherein the first blade is straight and positioned at a first acute angle relative to a centerline of the surgical cutting attachment in the first orientation, and the second blade is straight and positioned at a second acute angle relative to a centerline of the surgical cutting attachment in the first orientation.

3. The surgical cutting attachment of claim 1, wherein top sides of the first and second members are aligned together in the first orientation.

4. The surgical cutting attachment of claim 1, wherein the pivot is positioned between the first blade and the first set of apertures and also positioned between the second blade and the second set of apertures.

5. The surgical cutting attachment of claim 1, wherein each of the first and second blades includes a tapered shape that terminate at a tip, with the tips of the first and second members being in contact in the second orientation.

6. The surgical cutting attachment of claim 1, wherein the connector includes a pair of opposing wings that are pivotally connected to one of the second member and movable between an unlocked orientation and a locked orientation, the arms being positioned a greater distance from a centerline of the surgical cutting attachment in the unlocked orientation.

7. The surgical cutting attachment of claim 1, wherein a thickness of the first member varies along a length between the distal and proximal ends.

8. The surgical cutting attachment of claim 1, wherein the proximal end of the first member includes a ramp orientated at an acute angle relative to a centerline of the surgical cutting attachment and the proximal end of the second member includes a cavity, the inclined ramp being positioned in the cavity.

9. A surgical cutting attachment to cut an elongated member comprising:
a body with elongated first and second members each with distal and proximal ends and being pivotally connected together at a pivot between the distal and proximal ends, the first member positioned within a channel formed between spaced-apart first and second side walls of the second member, the body including a cutting blade at the distal ends of each of the first and second members, the body including a first aperture in the first member, a second aperture in the first side wall, and a third aperture in the second side wall, the third aperture being larger than the first and second apertures, the first and second apertures each including sharpened edges;
the first member being pivotally connected to the second member with the body movable between a first orientation with each of the first, second, and third apertures being aligned and the cutting blades being spaced apart, and a second orientation with the first and second apertures being out of alignment and the second and third apertures being in alignment, the second orientation also including the cutting blades being positioned together; and
the body further including a cavity formed by the proximal ends of the first and second members.

10. The surgical cutting attachment of claim 9, wherein the first member includes a ramp oriented transverse to a centerline of the body, the ramp being positioned and exposed within the cavity.

11. The surgical cutting attachment of claim 10, wherein the cavity is formed in the proximal end of the second member and the proximal end of the first member is recessed within an interior of the cavity.

12. The surgical cutting attachment of claim 11, further including a pair of wings pivotally attached to the second member and extending outward beyond the proximal end of the second member, the pair of wings movable between a locked position and an unlocked position.

13. The surgical cutting attachment of claim 9, wherein the second aperture is aligned at a first end of the third aperture in the first orientation and aligned with a second end of the third aperture in the second orientation.

14. The surgical cutting attachment of claim 9, wherein the first and second apertures each include a circular cross-sectional shape.

15. The surgical cutting attachment of claim 14, wherein the first aperture is smaller than the second aperture.

16. The surgical cutting attachment of claim 9, wherein widths of the channel and the first member vary along a length of the body between the proximal and distal ends.

17. A method of cutting an elongated member during a surgical procedure comprising:
positioning first and second elongated members in a first orientation with the first member positioned in a channel between side walls of the second member and contacting a bottom side of the first member against a bottom wall of the channel;
receiving the elongated member within a shear mechanism that includes a first aperture in the first member that aligns with a second aperture in the second member in the first orientation;
cutting the elongated member a first time with the shear mechanism by pivoting the first member relative to the second member to a second orientation and moving the first and second apertures out of alignment, the second orientation including the bottom side of the first member spaced away from the bottom wall of the channel;
receiving the elongated member within a scissor mechanism with the first and second members in the first orientation, the scissor mechanism including a first blade of the first member and a second blade of the second member that are spaced apart in the first orientation; and
cutting the elongated member a second time with the scissor mechanism by moving the first and second members from the first orientation to the second orientation and moving the first blade towards the second blade.

18. The method of claim 17, further comprising aligning a third aperture in the second member with the first aperture in the first member and the second aperture in the second member when positioning the first and second elongated members in the first orientation.

19. The method of claim 17, further comprising aligning a third aperture in the first member with a fourth aperture in the second member while aligning the first and second apertures.

20. The method of claim 17, further comprising aligning a top side of the first member with a top side of the second member when moving the first and second members to the first orientation.
